(19) United States
(12) Patent Application Publication
(10) Pub. No.: US 2004/0254247 A1
(43) Pub. Date: Dec. 16, 2004

(54) Method and composition for treatnment of ocular hypertension and glaucoma

(76) Inventor: Ryuji Ueno, Montgomery, MD (US)

Correspondence Address: SUGHRUE MION, PLLC, 2100 PENNSYLVANIA AVENUE, N.W., SUITE 800, WASHINGTON, DC, 20037, US

(21) Appl. No.: 10/485,370

(22) PCT Filed: Jul. 30, 2002
(86) PCT No.: PCT/JP02/07699

Related U.S. Application Data
(60) Provisional application No. 60/308,589, filed on Jul. 31, 2001.

(51) Int. Cl. A61K 31/557

(52) U.S. Cl. 514/573; 514/690

(57) ABSTRACT

The instant invention discloses a method for treatment of ocular hypertension and glaucoma, which comprises administrating topically to the eyes of a mammalian subject in need of such treatment more than 5 μg and less than 50 μg per eye per administration of 15-keto-prostaglandin compound having a ring structure at the end of the ω chain. The treatment of the present invention causes substantially no or reduced ophthalmic irritating side effect even in such a high dose.

### TECHNICAL FIELD

The present invention relates to a method for treating ocular hypertension and glaucoma, which causes reduced or substantially no ocular irritation such as conjunctival hyperemia. The present invention also provides a composition useful for treatment of the present invention.

### BACKGROUND ART

Prostaglandins (hereinafter referred to as PG(s)) are the members of class of organic carboxylic acids that are contained in the tissues or organs of humans or other mammals and exhibit a wide range of physiological activity. PGs found in nature (primary PGs) generally have a prostanoic acid skeleton as shown in the formula (A):

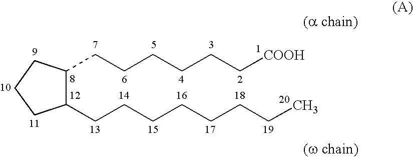

On the other hand, some of synthetic analogues of primary PGs have modified skeletons. The primary PGs are classified to PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGE₁, PGE₂ and PGE₃ are known to have vasodilation, hypotension, gastric secretion decreasing, intestinal tract movement enhancement, uterine contraction, diuretic, bronchodilation and anti ulcer activities. PGF_{1α}, PGF_{2α} and PGF_{3α} have been known to have hypertension, vasoconstriction, intestinal tract movement enhancement, uterine contraction, lutein body atrophy and bronchoconstriction activities.

Some 15-keto (i.e., having oxo at the 15-position instead of hydroxy)-PGs and 13,14-dihydro-15-keto-PGs are known as the substances naturally produced by the action of enzymes during the metabolism of primary PGs. It is also known that some 15-keto-PG compounds have intraocular pressure reducing effects and are effective for the treatment of ocular hypertension and glaucoma (U.S. Pat. Nos. 5,001,153, 5,151,444, 5,166,178 and 5,212,200, all of which are incorporated herein by reference).

Meanwhile, “Xalatan®” that has been launched as an eye drops for ocular hypertension and glaucoma contains, as an active ingredient thereof, latanoprost, i.e., 13,14-dihydro-17-phenyl-18,19,20-trinor-PGF_{2α} isopropyl ester, which is a prostaglandin derivative having a ring structure at the end of the ω chain and having hydroxy at the 15-position. The clinical concentration of latanoprost in the “Xalatan®” eye drops is 0.005% and, estimating from about 30-35 μl of one drop volume of “Xalatan®” eye drops, the clinical dose of latanoprost is about 1.5 μg-1.75 μg per eye per administration. Problematic side effects of this eye drops in clinically applied dose, including iris pigmentation, ocular irritation such as conjunctival hyperemia and chemosis of conjunctiva have been reported (American Journal of Ophthalmology 2001;131:631-635, Survey of Ophthalmology 1997; 41:S105-S110, the cited references are herein incorporated by reference).

It is known that a 15-keto-prostaglandin compound having a ring structure at the end of the ω chain has intraocular pressure reducing effects. U.S. Pat. No. 5,321,128 describes that administration of 13,14-dihydro-15-keto-17-phenyl-18,19,20-trinor-PGF_{2α} isopropyl ester to healthy human eyes and monkey eyes in a dose of 5 μg and 3 μg, respectively, showed intraocular pressure reducing effects, and showed no side effect such as conjunctival hyperemia, ocular irritation and foreign body sensation in the human. There is another document reporting that administration of 13,14-dihydro-15-keto-17-phenyl-18,19,20-trinor-PGF_{2α} isopropyl ester to monkey eyes (50 μg per eye) showed intraocular pressure reducing effects (Clinical Report Vol. 28, No. 11, pages 3505-3509, 1994).

However, in the treatment of ocular hypertension and glaucoma, nobody has known the extent of ocular irritation such as conjunctival hyperemia shown in the administration of a 15-keto-prostaglandin compound having a ring structure at the end of the ω chain to mammal eyes in a high dose.

### DISCLOSURE OF THE INVENTION

The present inventor has conducted intensive studies on the biological activity of a 15-keto-prostaglandin compound having a ring structure at the end of the ω chain, and found that administration of said compound topically to mammal eyes effectively lowered the intraocular pressure while causes substantially no or reduced ocular irritation such as conjunctival hyperemia, even in a high dose.

Namely, the present invention relates to a method for treatment of ocular hypertension and glaucoma, which comprises administrating a 15-keto-prostaglandin compound having a ring structure at the end of the ω chain topically to the eyes of a mammalian subject in need of such treatment more than 5 μg and less than 50 μg per eye per administration. According to the present invention, intra ocular pressure of the subject is effectively lowered while substantially no or reduced ocular irritation, such as conjunctival hyperemia, is observed despite of the high dose.

The present invention further relates to an ophthalmic composition for treating ocular hypertension and glaucoma of a mammalian subject, which comprises a 15-keto-prostaglandin compound having a ring structure at the end of the ω chain in an amount to provide a dose of more than 5 μg and less than 50 μg per eye per administration.

The present invention further relates to use of a 15-keto-prostaglandin compound having a ring structure at the end of the ω chain for manufacturing an ophthalmic composition for treating ocular hypertension and glaucoma of a mammalian subject, wherein said composition comprises the 15-keto-prostaglandin compound in an amount to provide a dose of more than 5 μg and less than 50 μg per eye per administration.

In the present invention, the “15-keto-prostaglandin compound” (hereinafter, referred to as “15-keto-PG compound”) may include any of derivatives or analogs (including substituted derivatives) of a compound having an oxo group at 15-position of the prostanoic acid skeleton instead of the hydroxy group, irrespective of the configuration of the five-membered ring, the number of double bonds, presence or absence of a substituent, or any other modification in the α or ω chain.

The nomenclature of the 15-keto-PG compounds used herein is based on the numbering system of the prostanoic acid represented in the above formula (A).

A preferred compound used in the present invention is represented by the formula (I):

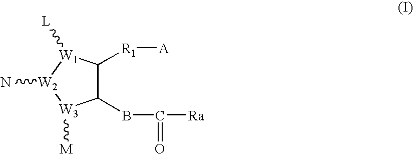

[wherein W₁, W₂ and W₃ are carbon atom or oxygen atom,

L, M and N are hydrogen, hydroxy, halogen, lower alkyl, hydroxy(lower)alkyl or oxo (wherein at least one of L and M is a group other than hydrogen, and the five-membered ring may have at least one double bond);

A is —CH₂OH, —COCH₂OH, —COOH or a functional derivative thereof;

B is —CH₂—CH₂—, —CH═CH— or —C≡C—;

R₁ is a saturated or unsaturated bivalent lower or medium aliphatic hydrocarbon residue, which is unsubstituted or substituted by halogen, alkyl, hydroxy, oxo, aryl or heterocyclic group; and

Ra is a saturated or unsaturated lower or medium aliphatic hydrocarbon residue, which is substituted at the end by cyclo(lower)alkyl, cyclo(lower)alkyloxy, aryl, aryloxy, heterocyclic group or hetrocyclic-oxy group.]

A group of particularly preferable compounds among the above-described compounds are represented by the formula (II):

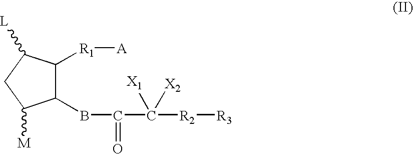

[wherein L and M are hydrogen, hydroxy, halogen, lower alkyl, hydroxy(lower)alkyl or oxo (wherein at least one of L and M is a group other than hydrogen, and the five-membered ring may have at least one double bond);

A is —CH₂OH, —COCH₂OH, —COOH or a functional derivative thereof;

B is —CH₂—CH₂—, —CH═CH—, —C≡C—;

X₁ and X₂ are hydrogen, lower alkyl, or halogen;

R₁ is a saturated or unsaturated bivalent lower or medium aliphatic hydrocarbon residue, which is unsubstituted or substituted with halogen, alkyl, hydroxy, oxo, aryl or heterocyclic group;

R₂ is a single bond or lower alkylene; and

R₃ is cyclo(lower)alkyl, cyclo(lower)alkyloxy, aryl, aryloxy, heterocyclic group or heterocyclic-oxy group.]

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 6 to 10 carbon atoms for R₁ and 1 to 10, especially 1 to 8 carbon atoms for Rₐ.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon-group containing 1 to 6 carbon atoms and includes, for example, methyl, ethyl, propyl, isopropyl, butyl, isobutyl, t-butyl, pentyl and hexyl.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo (lower) alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituent include halogen atom and halo substituted (lower) alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom(s) and 1 to 4, preferably 1 to 3 of 1 or 2 type of hetero atoms selected from nitrogen atom, oxygen atom and sulfer atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, lysine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl (lower) alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A means a group represented by the formula —CONR′R″, wherein each of R′ and R″ is hydrogen atom, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydroxy which provides a 5-membered ring structure of, so called, PGF type.

Preferred A is —COOH, its pharmaceutically acceptable salt, ester or amide thereof.

Preferred B is —CH₂—CH₂—, which provide the structure of so-called, 13,14-dihydro type.

Preferred example of X₁ and X₂ is that at least one of them is halogen, more preferably, both of them are halogen, especially, fluorine that provides a structure of, so called 16,16-difluoro type.

Preferred R₁ is a hydrocarbon containing 1-10 carbon atoms, preferably, 6-10 carbon atoms.

Examples of R₁ include, for example, the following groups:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C—C—CH₂—CH₂—CH₂—,

—CH₂—CH₂—CH₂—CH₂—CH (CH₃)—CH₂—

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—,

—CH₂—CH₂—CH₂—CH₂—CH₂—CH₂—CH₂ (CH₃) —CH₂—

Preferred Ra is a hydrocarbon containing 1-10 carbon atoms, more preferably, 1-8 carbon atoms which is substituted by aryl or aryloxy at the end.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

The typical example of the compound used in the invention is a 13,14-dihydro-15-keto-17-phenyl-18,19,20-trinor-prostaglandin F compound and its derivative or analogue.

The 15-keto-PG compound of the present invention may be in the keto-hemiacetal equilibrium by formation of a hemiacetal between hydroxy at position 11 and oxo at position 15.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the 15-keto-PG compounds used in the invention include both isomers. Further, while the compounds used in the invention may be represented by a structure formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485 (these cited references are herein incorporated by reference).

The term “treatment”, “treat” or “treating” used herein includes any means of control such as prevention, care, relief of the condition, attenuation of the condition, arrest of progression of the condition.

The term “a subject in need of such treatment” means a subject who is suffering from a disease in which a reduction in his/her intraocular pressure is desirable, for example, glaucoma and ocular hypertension, or a subject who is susceptible to suffering from such disease as discussed above. The subject may be any mammalian subject including human beings.

According to the present invention, the 15-keto-PG compound described as above may be formulated as an ophthalmic composition and applied topically to the eyes of a mammalian subject. The ophthalmic composition of the present invention may be any form for local eye administration used in the ophthalmic field such as eye drops and eye ointment. The ophthalmic composition may be prepared in a conventional manner known to the art. The eye drops may be prepared by dissolving the active ingredients in a sterile aqueous solution such as saline and buffering solution, or the eye drop composition may be provided as a combined powder composition comprising the active ingredient to be dissolved in the aqueous solution before use.

Eye drops such as the ones as described in EP-A-0406791 are preferably used in the present invention (the cited reference is herein incorporated by reference). If desired, additives ordinarily used in conventional eye drops maybe added. Such additives may include isotonizing agents (e.g., sodium chloride), buffering agent (e.g., boric acid, sodium monohydrogen phosphate, sodium dihydrogen phosphate), preservatives (e.g., benzalkonium chloride, benzethonium chloride, chlorobutanol), thickeners (e.g., saccharide such as lactose, mannitol, maltose; hyaluronic acid or its salt such as sodium hyaluronate, potassium hyaluronate; mucopolysaccharide such as chondroitin sulfate; sodium polyacrylate, carboxyvinyl polymer, crosslinked polyacrylate.)

The eye drops may be formulated as a sterile unit dose type eye drops containing no preservatives.

Eye ointment may also be prepared in a conventional manner known to the art. For example, it may be prepared by mixing the active ingredient into a base component conventionally used for known eye ointments under a sterile condition. Examples of the base components for the eye ointment include petrolatum, selen 50, Plastibase and macrogol, but not limited thereto. Further, in order to increase the hydrophilicity, a surface-active agent can be added to the composition. The eye ointment may also contain the above-mentioned additives such as the preservatives and the like, if desired.

According to the present invention, more than 5 μg and less than 50 μg per eye of the above-defined 15-keto-compound is topically administered to the subject per administration. The dose of the 15-keto-compound is preferably less than 30 μg, more preferably less than 20 μg, further more preferably less than 15 μg and still further preferably less than 12 μg per eye per administration. The lower limit of the dose may be more than 7 μg or more than 10 μg per eye per administration.

The dose of the above-defined 15-keto-compound may vary within the range defined as above depending on the compound to be used, the type of subject such as animals or human, age, weight, symptom to be treated, desirable therapeutic effect, period for treatment and the like.

According to the invention, the frequency of the administration of the above-defined 15-keto-prostaglandin compound may vary depending on the compound to be used, the type of subject such as animals or human, age, weight, symptom to be treated, desirable therapeutic effect, period for treatment and the like. The frequency of administration may be at least once a day and preferably, one to six times, more preferably, one to four times a day.

Since the 15-keto-prostaglandin compound used in the invention causes substantially no or reduced ocular irritation even in a high dose, the treatment of the instant invention may be carried out for a long period of time.

The ophthalmic composition of the invention may contain a single active ingredient or a combination of two or more active ingredients. In a combination of plural active ingredients, their respective contents may be suitably increased or decreased in consideration of their therapeutic effects and safety.

The concentration of the 15-keto-prostaglandin compound in the ophthalmic composition of the present invention is adjusted so that the amount of the compound to be administrated is within the range of more than 5 μg and less than 50 μg, preferably less than 30 μg, more preferably less than 20 μg, further more preferably less than 15 μg and still further preferably less than 12 μg per eye per administration. The lower limit of the amount may be more than 7 μg or more than 10 μg per eye per administration.

Further, the ophthalmic composition of the present invention may contain any other pharmaceutically active ingredients as far as they are not contrary to the objects of the present invention.

Despite the high dose of the 15-keto-prostaglandin compound, a safe and comfortable treatment of ocular hypertension and glaucoma can be conducted for a long period of time according to the present invention. Besides, the present ophthalmic composition may be administered safely to subjects with ocular hypertension and glaucoma having some disorders on their cornea or conjunctiva such as allergic disease and dry eye.

The present invention will be described in more detail with reference to the following example, which is not intended to limit the present invention.

### EXAMPLE 1

The incidence rate of conjunctival hyperemia was compared between the present compound 13,14-dihydro-15-keto-17-phenyl-18,19,20-trinor-PGF_{2α} isopropyl ester and 13,14-dihydro-17-phenyl-18,19,20-trinor-PGF_{2 α} isopropyl ester.

1) Method

Either 13,14-dihydro-15-keto-17-phenyl-18,19,20-trinor-PGF_{2α} isopropyl ester or 13,14-dihydro-17-phenyl-18,19,20-trinor-PGF_{2 α} isopropyl ester was ocularly administered once to one eye of white rabbits (three cases each, total of 12 cases) in a dose of 1.75 μg or 50 μg.

2) Evaluation Method

The presence of conjunctival hyperemia was examined at two hours after the administration and the ratio of cases showing conjunctival hyperemia was evaluated by percent in each group.

3) Results

Table 1 shows the results.

TABLE 1

Incidence Rate of
Conjunctival Hyperemia

| Groups | 1.75 μg eye drop | 50 μg eye drop |
|---|---|---|
| 13,14-dihydro-15-keto-17- | 0% | 33% |
| phenyl-18,19,20-trinor- | | |
| PGF_{2α} isopropyl ester | | |
| 13,14-dihydro-17-phenyl- | 67% | 100% |
| 18,19,20-trinor-PGF_{2α} | | |
| isopropyl ester | | |

In the administration of 13,14-dihydro-17-phenyl-18,19,20-trinor-PGF_{2α} isopropyl ester, 67% of the subjects receiving a clinical dose of 1.75 μg and 100% of the subjects receiving 50 μg showed conjunctival hyperemia.

On the other hand, in the administration of the present compound 13,14-dihydro-15-keto-17-phenyl-18,19,20-trinor-PGF_{2α} isopropyl ester, none of the subjects receiving the dose of 1.75 μg showed conjunctival hyperemia. Even in the administration of a high dose of 50 μg, the percent of the subjects showing conjunctival hyperemia was only about half the percent of the subjects receiving 1.75 μg of 13,14-dihydro-17-phenyl-18,19,20-trinor-PGF_{2α} isopropyl ester.

### EXAMPLE 2

Method

13,14-dihydro-15-keto-17-phenyl-18,19,20-trinor-PGF_{2α} isopropyl ester at a dose of 30 μg was ocularly administered to one eye of Dutch rabbits (4 male and 4 female, total 8 cases). The administration was performed 4 times a day with 2-hour intervals for 13 weeks. The cornea, conjunctiva and iris were observed about occurrence of irritable response before, and 4, 8, and 13 weeks after the initiation of the administration. The observation was performed within the time from 0.5 hour to 2 hours after the last administration of the day. The cornea was observed about a presence of opacity and its area in the cornea. The conjunctiva was observed about occurrence of hyperemia, redness and swelling. The iris was observed about occurrence of hyperemia, congestion, swelling and hemorrhage. The ratio of cases showing irritable response in the cornea, conjunctiva or iris was evaluated by percent in the group.

Results

As shown in Table 2, the ocular administration of 13,14-dihydro-15-keto-17-phenyl-18,19,20-trinor-PGF_{2α} isopropyl ester at a dose of 30 μg/eye each 4 times a day for 13 weeks had no effect on the cornea, conjunctiva and iris.

TABLE 2

| | | | Incidence rate of ocular | | | |
|---|---|---|---|---|---|---|
| | | | irritation | | | |
| | | | Time after the initiation of | | | |
| | | | ocular administration | | | |
| | | | | 4 | 8 | 13 |
| Test Compound | Dose | n | Pre | weeks | weeks | weeks |
| 13,14-dihydro-15- | 30 μg/eye | 8 | 0% | 0% | 0% | 0% |
| keto-17-phenyl- | 4 times/day | | | | | |
| 18,19,20-trinor- | for 13 weeks | | | | | |
| PGF_{2α} isopropyl | | | | | | |
| ester | | | | | | |

These results demonstrate that the present compound causes substantially no or reduced ocular irritation even in a high dose.

### EXAMPLE 3

Method

13,14-dihydro-15-keto-17-phenyl-18,19,20-trinor-PGF_{2α} isopropyl ester at a dose of 12 μg was ocularly administered to one eye (test eye) of 6 healthy volunteers. The vehicle solution was ocularly administered to the contralateral eye (control eye). Intraocular pressure (IOP) was measured by means of an applanation tonometer before, and 4, 6, and 12 hours after the administration. The IOP lowering effect of the test compound was evaluated based on difference in IOP between test eye and the control eye at each time point of IOP measurement.

Results

As shown in Table 3, the ocular administration of 13,14-dihydro-15-keto-17-phenyl-18,19,20-trinor-PGF_{2α} isopropyl ester at a dose of 12 μg lowered the IOP by 1.0, 0.8 and 1.1 mmHg at 4, 6, and 12 hours after the administration respectively as compared with the control eye.

TABLE 3

| | | | Intraocular pressure | | |
|---|---|---|---|---|---|
| | | | lowering effect | | |
| | | | Time after | | |
| | | | administration (hours) | | |
| Test compound | dose | n | 4 | 6 | 12 |
| 13,14-dihydro-15-keto-17- | 12 | 6 | −1.0 | −0.8 | −1.1 |
| phenyl-18,19,20- | μg/eye | | mmHg | mmHg | mmHg |
| trinor-PGF_{2α} | | | | | |
| isopropyl ester | | | | | |

### EXAMPLE 4

Method

Nine male cynomolgus monkeys (body weights of animals ranged between 3.2 and 5.4 kg) without abnormalities in the anterior segment of the eye were used.

13,14-dihydro-15-keto-17-phenyl-18,19,20-trinor-PGF_{2α} isopropyl ester at a dose of 15 μg/eye was administered topically to the right eyes of the animals at an administration volume of 30 μL/eye. The left eye received an equal volume of the vehicle.

Intraocular pressure (IOP) measurements were conducted by means of an applanation pneumatonometer (Model 30 Classic™, Mentor O & O, Inc., USA) immediately before the administration (0-hour), and at 2, 4, 8, 12, 24, 28, and 32 hours after the administration.

Eye irritancy was scored according to the criteria presented in Table 4 at the same time points as IOP measurements. These criteria are based on those of Draize test.

TABLE 4
Scale for Scoring Ocular Lesions

| | | |
|---|---|---|
| 1. | Cornea | |
| A. | Opacity-Degree of Density (area which is most dense is taken | |
| | for reading.) | |
| | No opacity | 0 |
| | Scattered or diffuse area-details of iris clearly visible | 1 |
| | Easily discernible translucent areas, details of iris slightly | 2 |
| | obscured | |
| | Opalescent areas, no details of iris visible, size of pupil barely | 3 |
| | discernible | |
| | Opaque, iris invisible | 4 |
| B. | Area of Cornea Involved | |
| | One quarter (or less) but not zero | 1 |
| | Greater than one quarter-less than one-half | 2 |
| | Greater than one-half less than three quarters | 3 |
| | Greater than three quarters up to whole area | 4 |
| | Score equals A × B × 5 Total maximum = 80 | |
| 2. | Iris | |
| A. | Values | |
| | Normal | 0 |
| | Folds above normal, congestion, swelling, circumcorneal | 1 |
| | injection (any one or all of these or combination of any thereof) | |
| | Hemorrhage; gross destruction (any one or both of these) | 2 |
| | Score equals A × 5 Total maximum = 80 | |
| 3. | Conjunctivae | |
| A. | Redness (refers to palpebral conjunctivae only) | |
| | Normal | 0 |
| | Vessels definitely injected above normal | 1 |
| | More diffuse, deeper crimson red, individual vessels not easily | 2 |
| | discernible | |
| | Diffuse beefy red | 3 |
| B. | Chemosis | |
| | No swelling | 0 |
| | Any swelling above normal (includes nictitating membrane) | 1 |
| | Obvious swelling with partial eversion of the lids | 2 |
| | Swelling with lids about half closed | 3 |
| | Swelling with lids about half closed to completely closed | 4 |
| C. | Discharge | |
| | No discharge | 0 |
| | Any amount different from normal (does not include small | 1 |
| | amount observed in inner canthus of normal animals) | |
| | Discharge with moistening of the lids and hairs just adjacent to | 2 |
| | the lids | |
| | Discharge with moistening of the lids and considerable area | 3 |
| | around the eye Score (A + B + C) × 2 Total maximum = 20 | |

The maximum total score is the sum of all scores obtained for the cornea, iris and conjunctivae.

Results

As shown in Table 5, topical administration of 13,14-dihydro-15-keto-17-phenyl-18,19,20-trinor-PGF_{2α} isopropyl ester at a dose of 15 μg/eye produced a significant IOP reduction.

As shown in Table 6, no signs or symptoms of ocular irritation were observed after topical administration of 13,14-dihydro-15-keto-17-phenyl-18,19,20-trinor-PGF_{2α} isopropyl ester.

These results demonstrate that the compound used in the invention shows IOP-lowering effect while causes substantially no or reduced ocular irritation even in a high dose.

TABLE 5
Effects of 13,14-dihydro-15-keto-17-phenyl-18,19,20-trinor-PGF_{2α} isopropyl ester
on IOP in monkey eyes

| | IOP (mmHg) | | | | | | | |
|---|---|---|---|---|---|---|---|---|
| | Time after administration (hr) | | | | | | | |
| Treatment | 0 | 2 | 4 | 8 | 12 | 24 | 28 | 32 |
| Left eye: | 19.3 ± 0.4 | 19.8 ± 0.3 | 20.1 ± 0.3 | 20.1 ± 0.4 | 18.2 ± 0.5 | 19.4 ± 0.6 | 19.4 ± 0.4 | 18.7 ± 0.4 |
| Vehicle | | | | | | | | |
| Right eye: | 19.7 ± 0.6 | 17.7** ± 0.5 | 7.0** ± 0.5 | 16.6** ± 0.6 | 14.4** ± 0.8 | 16.6^{##} ± 0.6 | 16.7** ± 0.5 | 17.2* ± 0.4 |
| 13,14-dihydro-15- | | | | | | | | |
| keto-17-phenyl- | | | | | | | | |
| 18,19,20-trinor- | | | | | | | | |
| PGF_{2α} | | | | | | | | |
| isopropyl ester | | | | | | | | |
| (15 μg/eye) | | | | | | | | |

n = 9, Mean ± SE,
**p < 0.01,
*p < 0.05 compared to vehicle-treated contralateral eye (paired Student's t-test),
^{##}p < 0.01,
^{#}p < 0.05 compared to vehicle-treated contralateral eye (paired Wilcoxon's test)

TABLE 6
Evaluation for eye irritancy

| | | Eye irritancy (Score) | | | | |
|---|---|---|---|---|---|---|
| | Time after | Cornea | Iris | Conjunctivae | | |
| Treatment | administration | Opacity | Values | Redness | Chemosis | Discharge |
| 13,14-dihydro- | Before | 0 | 0 | 0 | 0 | 0 |
| 15-keto-17-phenyl- | 2 hr | 0 | 0 | 0 | 0 | 0 |
| 18,19,20-trinor- | 4 hr | 0 | 0 | 0 | 0 | 0 |
| PGF_{2α} isopropyl ester | 8 hr | 0 | 0 | 0 | 0 | 0 |
| (15 μg/eye) | 12 hr | 0 | 0 | 0 | 0 | 0 |
| | 24 hr | 0 | 0 | 0 | 0 | 0 |
| | 28 hr | 0 | 0 | 0 | 0 | 0 |
| | 32 hr | 0 | 0 | 0 | 0 | 0 |

Eye irritancy was scored according to the criteria presented in Table 4

1. A method for treatment of ocular hypertension and glaucoma, which comprises administrating topically to the eyes of a mammalian subject in need of such treatment more than 5 μg and less than 50 μg per eye per administration of a 15-keto-prostaglandin compound having a ring structure at the end of the ω chain.

2. The method as described in claim 1, wherein said 15-keto-prostaglandin compound is a compound represented by the following general formula (I): 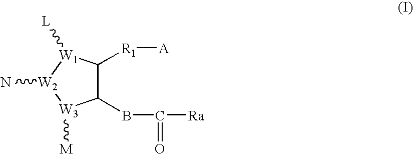
wherein W₁, W₂ and W₃ are carbon atom or oxygen atom,
L, M and N are hydrogen, hydroxy, halogen, lower alkyl, hydroxy(lower)alkyl or oxo provided that at least one of L and M is a group other than hydrogen, and the five-membered ring may have at least one double bond;
A is —CH₂OH, —COCH₂OH, —COOH or a functional derivative thereof;
B is —CH₂—CH₂—, —CH═CH— or —C≡C—;
R₁ is a saturated or unsaturated bivalent lower or medium aliphatic hydrocarbon residue, which is unsubstituted or substituted by halogen, alkyl, hydroxy, oxo, aryl or heterocyclic group; and
Ra is a saturated or unsaturated lower or medium aliphatic hydrocarbon residue, which is substituted at the end by cyclo (lower) alkyl, cyclo (lower) alkyloxy, aryl, aryloxy, heterocyclic group or hetrocyclic-oxy group.

3. The method as described in claim 1, wherein said 15-keto-prostaglandin compound is a 13,14-dihydro-15-keto-prostaglandin compound.

4. The method as described in claim 1, wherein said 15-keto-prostaglandin compound is a 13,14-dihydro-15-keto-17-phenyl-18,19,20-trinor-prostaglandin compound.

5. The method as described any one of claims 1-4, wherein more than 5 μg and less than 30 μg of said 15-keto-prostaglandin compound is administrated per eye per administration.

6. The method as described in claim 5, wherein more than 5 μg and less than 15 μg of said 15-keto-prostaglandin compound is administrated per eye per administration.

7. An ophthalmic composition for treatment of ocular hypertension and glaucoma of a mammalian subject, which comprises a 15-keto-prostaglandin compound having a ring structure at the end of the ω chain in an amount to provide a dose of more than 5 μg and less than 50 μg per eye per administration.

8. The composition as described in claim 7, wherein said 15-keto-prostaglandin compound is a compound represented by the following general formula (I): 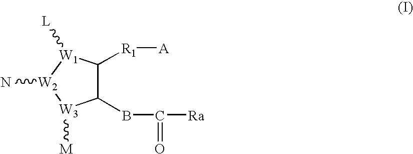
wherein W₁, W₂ and W₃ are carbon atom or oxygen atom,
L, M and N are hydrogen, hydroxy, halogen, lower alkyl, hydroxy(lower)alkyl or oxo provided that at least one of L and M is a group other than hydrogen, and the five-membered ring may have at least one double bond;
A is —CH₂OH, —COCH₂OH, —COOH or a functional derivative thereof;
B is —CH₂—CH₂—, —CH═CH— or —C≡C—;
R₁ is a saturated or unsaturated bivalent lower or medium aliphatic hydrocarbon residue, which is unsubstituted or substituted by halogen, alkyl, hydroxy, oxo, aryl or heterocyclic group; and
Ra is a saturated or unsaturated lower or medium aliphatic hydrocarbon residue, which is substituted at the end by cyclo (lower) alkyl, cyclo (lower) alkyloxy, aryl, aryloxy, heterocyclic group or hetrocyclic-oxy group.

9. The composition as described in claim 7, wherein said 15-keto-prostaglandin compound is a 13,14-dihydro-15-keto-prostaglandin compound.

10. The composition as described in claim 7, wherein said 15-keto-prostaglandin compound is a 13,14-dihydro-15-keto-17-phenyl-18,19,20-trinor-prostaglandin compound.

11. The composition as described in any one of claims 7-10, wherein the dose is more than 5 μg and less than 30 μg per eye per administration.

12. The composition as described in claim 11, wherein the dose is more than 5 μg and less than 15 μg per eye per administration.

13. The composition as described in any one of claims 7-12, wherein the ophthalmic composition is an eye drop composition.

14. Use of a 15-keto-prostaglandin compound having a ring structure at the end of the ω chain for manufacturing an ophthalmic composition for treating ocular hypertension and glaucoma of a mammalian subject, wherein said composition comprises a 15-keto-prostaglandin compound in an amount to provide a dose of more than 5 μg and less than 50 μg per eye per administration.

15. The use as described in claim 14, wherein said 15-keto-prostaglandin compound is a compound represented by the following general formula (I): 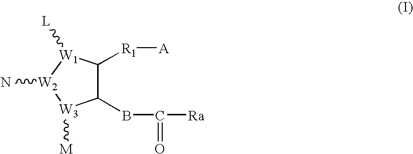
wherein W₁, W₂ and W₃ are carbon atom or oxygen atom,
L, M and N are hydrogen, hydroxy, halogen, lower alkyl, hydroxy(lower)alkyl or oxo provided that at least one of L and M is a group other than hydrogen, and the five-membered ring may have at least one double bond;
A is —CH₂OH, —COCH₂OH, —COOH or a functional derivative thereof;
B is —CH₂—CH₂—, —CH═CH— or —C≡C—;
R₁ is a saturated or unsaturated bivalent lower or medium aliphatic hydrocarbon residue, which is unsubstituted or substituted by halogen, alkyl, hydroxy, oxo, aryl or heterocyclic group; and
Ra is a saturated or unsaturated lower or medium aliphatic hydrocarbon residue, which is substituted at the end by cyclo (lower) alkyl, cyclo (lower) alkyloxy, aryl, aryloxy, heterocyclic group or hetrocyclic-oxy group.

16. The use as described in claim 14, wherein said 15-keto-prostaglandin compound is a 13,14-dihydro-15-keto-prostaglandin compound.

17. The use as described in claim 14, wherein said 15-keto-prostaglandin compound is a 13,14-dihydro-15-keto-17-phenyl-18,19,20-trinor-prostaglandin compound.

18. The use as described in any one of claims 14-17, wherein the dose is more than 5 μg and less than 30 μg per eye per administration.

19. The use as described in claim 18, wherein the dose is more than 5 μg and less than 15 μg per eye per administration.

20. The use as described in any one of claims 14-19, wherein the ophthalmic composition is an eye drop composition.
